(19) United States
(12) Patent Application Publication
(10) Pub. No.: US 2021/0282739 A1
(43) Pub. Date: Sep. 16, 2021

(54) STETHOSCOPE DEVICE AND METHOD FOR REMOTE PHYSICAL EXAMINATION OF A PATIENT

(71) Applicants: AVIHAI ESHEL, KIRIAT TIVON (IL); GUY KATZNELSON, TIVON (IL)

(72) Inventors: AVIHAI ESHEL, KIRIAT TIVON (IL); GUY KATZNELSON, TIVON (IL)

(21) Appl. No.: 17/202,440

(22) Filed: Mar. 16, 2021

Related U.S. Application Data
(60) Provisional application No. 62/989,909, filed on Mar. 16, 2020.

(51) Int. Cl. A61B 7/04; A61B 7/00; A61B 5/00; G16H 80/00; G16H 40/67

(52) U.S. Cl. CPC A61B 7/04; A61B 7/003; A61B 5/0004; A61B 2560/0214; A61B 5/6803; G16H 80/00; G16H 40/67; A61B 5/7405

(57) ABSTRACT

Devices and methods for remote physical examination of a patient are provided. A stethoscope device connectable to an external communication device is described. The stethoscope device includes a stethoscope head formed for placing directly on skin of a patient, a microphone to obtain physiological sounds and convert the sounds to electrical signals, a hollow tube to couple the stethoscope head to the microphone, a plug connectable to an external communication device, and a headset, speaker, or a headset port.

### FIELD

Embodiments of the invention relate generally to remote physical examination of a patient, especially auscultation of breathing, heart and other sounds.

### BACKGROUND

In current times, both doctors and patients run into a need for providing remote assistance and an initial diagnosis over the phone. In some cases, a patient may not be able to leave his residence for a medical check or examination, and a physician may wish to perform a remote examination, or obtain real time examination data without the need to perform a house visit. Patients who live in rural remote areas and require to travel long distances to meet their physician, others on travel, or in isolated locations with no physical access, such as in disaster areas or humanitarian crisis events, may require an examination by a physician from a remote location.

### SUMMARY

According to embodiments of the invention, a device for remote physical examination of a patient is provided. The stethoscope device includes a stethoscope head formed for placing directly on skin of a patient and adapted to capture physiological sounds, a microphone to receive the physiological sounds and/or voice sounds from the stethoscope head and convert the sounds to electrical signals, a hollow tube to couple the stethoscope head to the microphone, a plug connectable to an external communication device and a headset (e.g. an earpiece which is wired or wireless, or a speaker) or a headset port which is connectable to an external earpiece, speaker or headset. The stethoscope head may be adapted to obtain vocal sounds, e.g., emitted by the patient, in addition to the physiological sounds collected via the stethoscope head. The stethoscope device may further comprise a ring surrounding the stethoscope head. In some embodiments, a disposable cover may be provided, which is mountable on the stethoscope head for a single-time use. The stethoscope device may include an insulation layer which acoustically insulates the microphone from external audible interference. In some embodiments, the plug is connectable to an audio port of the external communication device, such as a mobile phone or mobile communication device. The device may comprise a power supply unit coupled to the microphone, and may include a housing adapted to enclose the power supply unit and the microphone. The microphone may be a microphone with an analog output. The wireless communication unit of stethoscope device may comprise an antenna and a power supply unit such as a battery, to provide power supply to various electronic components of the stethoscope device, e.g., to the microphone, an external speaker, a wireless controller, a LED indicator, etc.

According to another embodiment, a stethoscope device for remote physical examination of a patient comprises a stethoscope head formed for placing directly on skin of a patient and adapted to capture physiological sounds, a microphone to receive the physiological sounds from the stethoscope head and convert the sounds to electrical signals, a hollow tube configured to couple the stethoscope head to the microphone and a wireless communication unit to wirelessly connect the stethoscope device to an external communication device. The device may comprise a headset or headset port. An insulation layer which acoustically insulates the microphone from external audible interference may be included. The wireless communication unit comprises an antenna, a power supply unit, and a wireless controller, wherein the wireless controller is connected to the microphone, the antenna, and the power supply unit for wirelessly connecting the stethoscope head to an external device. The stethoscope device may include an amplifier to amplify obtained examination data.

According to another embodiment, a stethoscope device for remote physical examination of a patient comprises a stethoscope head formed for placing directly on skin of a patient, a microphone to obtain physiological sounds and convert the sounds to electrical signals, a hollow tube to couple the stethoscope head to the microphone, a power supply to provide power to the stethoscope device, and a speaker (or, alternatively, an earpiece or headset).

According to another embodiment, a method for remote physical examination of a patient comprises providing a stethoscope device comprising a stethoscope head formed for placing directly on skin of a patient, a microphone to obtain physiological sounds and convert the sounds to electrical signals, a hollow tube to couple the stethoscope head to the microphone, a plug connectable to an external communication device and a headset or a headset port. The method may include connecting the stethoscope device to a patient's external communication device, e.g., using a physical wire plug or via a wireless connection, and initiating a voice or video call between the patient's external communication device and a physician's external communication device (e.g., each side of the call using his/her mobile phone). The method further includes transmitting, in real time, physiological examination data obtained by the stethoscope device from the patient's external communication device to the physician's external communication device via the call, and receiving the physiological examination data by the physician's external communication device. The physician receiving the examination data may explain to the patient how to position the stethoscope device, and may ask for further examination data. The physician may perform a diagnosis of the patient's condition based on the received examination data, which includes physiological sound data and the patient's voice.

According to another embodiment, a method for remote physical examination of a patient comprises providing a stethoscope device comprising a stethoscope head formed for placing directly on skin of a patient, a microphone to obtain physiological sounds and convert the sounds to electrical signals, a hollow tube to couple the stethoscope head to the microphone, the stethoscope device configured to connect to an external communication device by a physical or a wireless connection. The method comprises using the external communication device, recording the physiological sounds obtained by the microphone, and storing the recording for use by a physician. Additional embodiments are applicable, as may be described further hereinbelow.

### BRIEF DESCRIPTION OF THE DRAWINGS

Disclosed herein is a stethoscope device which is connectable to a mobile device, e.g., a mobile phone. The stethoscope device may allow a patient or someone who is caring for the patient to perform a live medical examination, while the physician is speaking to the patient over the phone in a video or a voice call. The stethoscope device may be connectable (e.g., pluggable) to a mobile device, thus allowing performing a live video or voice communication with a physician, while performing a live remote medical examination, either a self-examination by the patient himself or examination which may be assisted by another person.

The examination data is output as audio data in a voice call or a video call, and is audible through a headset (e.g., one or two earpieces) or a speaker or wireless earpiece (such as Bluetooth earpiece) in the external communication device on the remote side of the call. Alternatively, in an asynchronous examination mode, the examination data may be recorded and stored (for example either in a local storage unit on the mobile device which is recording the data, or remotely using a cloud storage service) and later transmitted to or accessed by a physician.

Having thus described embodiments of the invention in general terms, reference will now be made to the accompanying drawings, which are not necessarily drawn to scale, and wherein:

FIG. 1 illustrates a block diagram of an example stethoscope device for performing a remote physical examination of a patient according to one embodiment of the invention;

FIG. 2 illustrates a block diagram of an example device for performing a remote physical examination of a patient according to a second embodiment of the invention;

FIGS. 3, 4 and 5 are flowcharts of methods for remote examination of a patient according to embodiments of the invention;

FIG. 6A is an exemplary diagram of a top view of a Printed Circuit Board (PCB) in accordance with an embodiment of the invention;

FIG. 6B is an exemplary diagram of a bottom view of a Printed Circuit Board (PCB) in accordance with an embodiment of the invention; and

FIG. 7 is a diagram of an example stethoscope device for performing a remote physical examination of a patient according to another embodiment of the invention.

Similar components are marked by the same numeral throughout the figures.

### DETAILED DESCRIPTION

Example embodiments will now be described more fully hereinafter with reference to the accompanying drawings, in which some, but not all, embodiments are shown. Indeed, the embodiments may take many different forms and should not be construed as limited to the embodiments set forth herein; rather, these embodiments are provided so that this disclosure will satisfy applicable legal requirements. Like reference numerals refer to like elements throughout.

Many situations arise in which patients cannot come to a physical examination at a physician's clinic, or alternatively the physician may not perform a home examination of the patient. For example, if the patient is in a remote location, or may have a very infectious disease in which case it is not advisable to go to a doctor's clinic, e.g., during a pandemic when a quarantine or lockdown is in effect. A physician may wish to perform a remote physiological examination, for example listening to the patient's lungs, heart, or abdomen.

A typical stethoscope comprises rubber tubing, and a stethoscope head with either a diaphragm (formed as a disc, e.g., made of plastic or other material) or a bell (formed as hollow cup, e.g., made of metal). The diaphragm accentuates certain sounds, for example high-frequency sounds; the bell transmits low-frequency sounds. Some stethoscopes are dual-headed, having both a diaphragm and a bell. Some stethoscopes have only a diaphragm (no bell); some stethoscopes have a pressure-sensitive tunable head that functions as both a diaphragm and bell depending on the applied pressure. The goal is for the physiological examination sound data to be transmitted from the patient's body to the eardrum of the physician or medical examiner, through an unbroken system.

The inventors have found that physiological sounds can be picked up using a local microphone, and transmitted through a mobile device such as a mobile phone, either in real time using a voice or a video call set up between the patient and the examining physician. The phone or video call serves another purpose—it enables the patient to be in direct communication with the physician during the remote examination, and allows the physician to explain where to position the stethoscope bell in order to perform the examination properly. In case of a video call, it also allows the physician to see where the patient (or someone assisting the patient) places the stethoscope, and to physically show the patient how and where to place the stethoscope. Corrections such as different placements of the stethoscope cell or the amount of pressure applied by the patient to the stethoscope bell can easily be made by the physician during the examination call.

It is noted that the remote examination need not necessarily be performed in real time. Instead, in an asynchronous mode, the examination data may be recorded by the patient (or a person assisting the patient) using the tele-stethoscope device, and the data may be sent (e.g., via email or other file transfer method) to the examining physician. The physician may review the recorded examination data, and provide a diagnosis based on it. Recording can also be performed using another recording device into which the pluggable stethoscope device may be connected, for example if the stethoscope device is connected to a SIM-based medical equipment device that includes recording capability.

For the asynchronous mode, written instructions detailing how to use the stethoscope, e.g., where to place it during the medical examination (for the purpose of recording), may be included in a user kit.

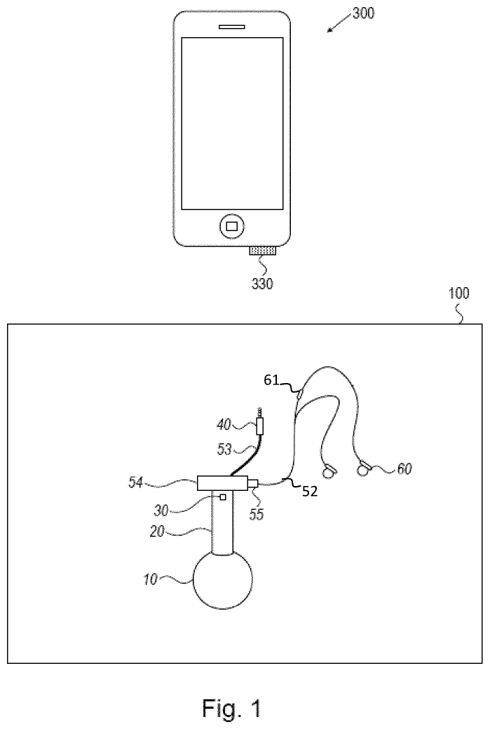

Reference is now made to FIG. 1, which illustrates a block diagram of an example tele-stethoscope device 100 for performing a remote physical examination of a patient according to one embodiment of the invention.

The stethoscope device 100 may include or otherwise be in communication with a stethoscope head 10, e.g., formed as a bell or a cup for placing directly on the skin of a patient and for passing the audio signals from the patient's body. It is noted that in some embodiments, the stethoscope head 10 may be or may include a diaphragm. In the embodiment of a bell which does not include a diaphragm, the stethoscope head 10 may be formed with a sufficient curvature, e.g., not too flat, that by placing it on the patient's skin enables picking up the physiological sounds during an examination in an optimal manner. If the curvature of the bell is insufficient, it may pick up noise that originates from the bell contacting the skin, in addition to the internal physiological sounds, thus may cause the examination data to be too noisy or corrupt to allow a physician to perform a proper diagnosis based on it.

Stethoscope device 100 further includes a microphone 30 for receiving the physiological sounds passed from stethoscope head 10. The microphone 30 may be operationally connected, e.g., via a cable (or a splitter unit such as splitter 54) to a plug 40. The microphone 30 may be a high sensitivity or ultra-high sensitivity microphone with high signal to noise ratio. The microphone may be of a relatively small size, adapted to fit into a tubing 20 which may be approximately the diameter of 5-10 mm, e.g., a tube diameter of 6 mm.

The physiological sound data obtained during a medical examination made on a part of a human body where the stethoscope device is positioned, may be converted into analog electrical sound data by microphone 30. When a phone call or video call is initiated between the patient and the physician, the examination data which is obtained on the patient's side includes both the physiological sound data received by microphone 30, and the patient's voice which is also obtained by the same microphone 30. Both these audio inputs are merged by microphone 30 into a single examination data stream which may be either recorded and stored, and/or transmitted from the communication device 300 to the remote device participating the call, e.g., to the physician's communication device. Examples of external communication devices (e.g., device 300) appropriate for connecting to the stethoscope device may include mobile phones, tablets, computers connectable to a network, or any other communication device that enables setting up a call via telephony and/or IP networks.

Alternatively, the patient's voice may be obtained by a separate microphone which may be connected to headset 60. The transmission may occur over any network infrastructure, e.g., over a telephony network, or over the Internet using any network or combination of networks such as a cellular network, wired computer network such as LAN, wireless network such as a WIFI network, telephony network, or satellite network. In some embodiments, the network may be a combination of several network types. It is noted that low latency end-to-end network connections would likely provide a higher quality live examination data.

The analog sound signal output by microphone 30 may be converted to digital signal by the external communication device 300 to which the stethoscope 100 is connected. The microphone may deliver analog electrical sound signals. In some cases, digital sound of the heart and lungs may sound different than using a device that captures analog sound. Physicians using a digital stethoscope may need to train for diagnosing a patient's state based on the digitized sounds.

In one embodiment, the examination data analyzed by the doctor will be based on the analog sound, which is digitally encoded by the external communication device for transmission over the underlying network, then decoded by the remote device, e.g., the device situated at the physician's side. As a result, the physician may listen to the natural sound of the physiological examination rather than a digitally filtered sound which is obtained when using a microphone with digital output. Similarly, in case of asynchronous examination, the examination data may be recorded using the analog sound data.

Microphone 30 may be selected from a myriad of analog microphones which provide analog audio output, e.g.: a MEMS microphone, a condenser such as an electret microphone which is a type of electrostatic capacitor-based microphone, a dynamic microphone, a ribbon microphone, a carbon microphone, a piezoelectric microphone, a fiber-optic microphone, a laser microphone, a liquid microphone. Such analog microphones may be manufactured by various manufacturers such as TDK, Brauner, CUI Devices, Sony, Olympus, etc. In other embodiments, the microphone 30 may have a digital output.

The microphone 30 may be operated in an operating voltage that is less than the operating voltage enabled by the external communication device, e.g., less than 1.8V if the mobile device 300 is limited to 1.8V. For example, an operating voltage of 1.65V may be used in case of a 1.8V limitation of the external device. N some embodiments, the microphone 30 may be an omni-directional microphone with a high sensitivity that is between, e.g., −100 dB to −20 dB at 1 kHz.

It is noted that in the embodiment of a bell included in the stethoscope head 10, the stethoscope head 10 may comprise a surrounding ring. The ring may be made of a relatively soft material, e.g., soft rubber or silicon, configured to dampen environmental noise and to stop the stethoscope head 10 from vibrating, making the stethoscope head 10 less prone to static electrical noise or external noise interference. In some embodiments, stethoscope head 10 may be coverable with a disposable cover which is mountable on the stethoscope bell for a single-time use. The disposable cover may be made of single-use nylon, fabric, paper, plastic, etc.

Stethoscope device 100 includes tubing 20, which comprises a hollow tube that delivers physiological sounds emitted from a patient's body (e.g., heartbeat, lung inflation and deflation, breathing sounds, arterial bruits, etc.) and picked up by the stethoscope head 10, to microphone 30. The stethoscope and the microphone 30 are adapted to pick up the voice of a patient (and/or of another person who may be assisting the patient and speaking to the physician), in addition to the physiological sounds that are picked up by stethoscope head 10 and delivered to the microphone 30 via the tubing 20.

In some embodiments, the microphone 30 may be operationally connected to a dedicated power supply such as a battery, which allows it to receive a steady and consistent supply of power which enables an optimal working point for the microphone. It is noted that adding an external power supply may be important for achieving an appropriate stability of the microphone for obtaining high-quality audio signal of the sampled physiological audio data, thus enabling proper diagnosis by the examining physician who is listening to the audio data transmitted over the phone or video call. An on-off switch may be added to the stethoscope device 100, configured to connect the power supply to the microphone 30 or disconnect it. In some embodiments, the power supply unit may provide power to additional components of the stethoscope device 100, e.g., to the microphone 30, to a connected speaker, to a wireless communication unit, etc.

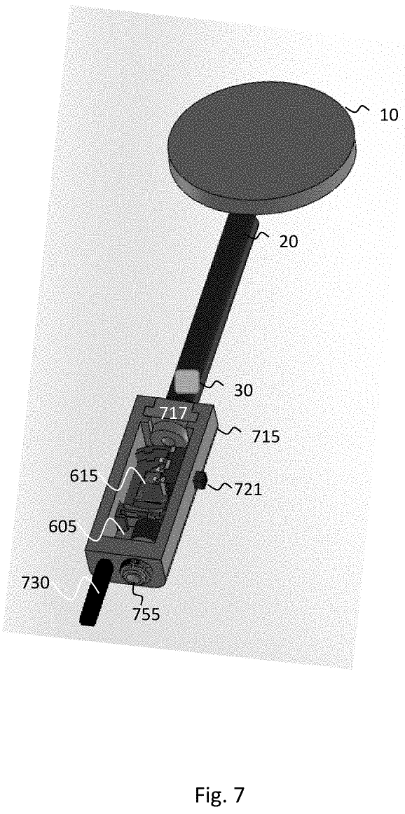

The tubing 20 may include, or may be coupled to, a housing according to FIG. 7. The tubing 20 is also useful for acoustic insulation of the microphone 30 from the stethoscope head 10 and the surrounding environment, while the stethoscope head 10 is in contact with a patient's skin. Tubing 20 may also provide an accentuation of the physical examination sounds. Tubing 20 may have a certain length, e.g., a minimal length that is on one hand convenient for a patient to hold and move according to the physician's instructions, to position directly on the skin at locations that the physician indicates. On the other hand, the tubing 20 should not be too long, so as not to cause reduction or attenuation of the physiological audio signals that are picked up by the stethoscope head 10 and passed onto the microphone. Tubing 20 may be made of plastic, rubber or other appropriate material. The stethoscope device 100 may comprise one or more amplifiers, for example, microphone 30 may be optionally coupled to an amplifier (not shown), which amplifies the audio output of the microphone 30. Such amplifier may amplify the physiological sounds before the signals from microphone 30 are delivered to external communication device 300. A second amplifier may be included in device 100, the second amplifier may be configured to amplify the sounds that the patient hears (e.g., the physician's voice transmitted via the call) —the second amplifier would assist hearing, for example for a hearing-impaired person while using device 100 in a live examination with a physician who is in a remote location.

Microphone 30 may optionally be insulated with an insulation layer 32, using acoustic insulating material such as foam, rubber, etc. The insulation layer 32 may be configured to surround (e.g., either substantially or partially surround) the microphone 30, or to surround tubing 20 or a portion thereof. The tubing may be of a relatively short length, e.g., between 1-30 cm. In one embodiment, the tubing may be of approximately 5-10 mm in diameter and may be configured for appropriate acoustic impedance matching.

Stethoscope device 100 may include a plug 40 which may be plugged into an audio port or audio connector of an external communication device 300, e.g., a USB-type connector (e.g., Micro-USB, Mini-USB, USB-A, USB-B, USB-C), a power connector, or any other type of connector, which is configured to be connected to a port of external communication device 300.

It is noted that in some embodiments, an adapter may be used to adapt the form factor of plug 40 to a socket/port form factor of an external communication device, e.g., to an audio port 330 of a mobile phone, a power port, or to a USB port of a tablet or a personal computer.

A connector or splitter 54 may be included in stethoscope device 100. The splitter 54 may include a connector that connects between: (a) headset port 55, or wire/cable 52 leading to the earpiece or headset 60, (b) cable 53 which leads to the plug 40, and (c) microphone 30 which may be coupled to the edge of tubing 20 or situated inside the tubing. In one embodiment, splitter 54 may reside in a dedicated housing or enclosure which includes headset port 55, microphone 30 and the connection to cable 53. Optionally, the tubing 20 or a portion thereof may be included in the housing. The splitter 54 allows the physiological sound data obtained by the stethoscope head 10 to be delivered from the microphone 30 to the audio input port 330 of external communication device 300, and from device 300 the sound data may be transmitted as a standard video or voice call which may be initiated with a physician's external communication device. In some embodiments, splitter 54 may further comprise or may be coupled to an amplifier for amplifying the sound data that is passed via the microphone 30. Microphone 30 may optionally be insulated, using acoustic insulating material such as foam, etc. the insulation layer may be configured to substantially surround or cover the microphone 30.

Stethoscope device 100 may include a headset port 55 or an earpiece jack/port, which is connectable to an external wired or wireless earpiece (e.g., Bluetooth earpiece), headset or speaker. Additionally or alternatively, stethoscope device 100 may include at least one earpiece or headset 60, which may be directly connected to the plug 40. Headset 60 may include one or two earpieces, and in some embodiments may include a microphone 61, which may be configured to pick up the patient's voice during the examination. The headset 60 delivers the voice sounds of the voice or video call that is set up between the patient and the physician, which is required for providing the physician's instructions during the remote physical examination. Headset 60 and is connected to the plug 40 via cable 52, which may be a shielded or insulated cable.

Stethoscope device 100 includes a plug 40 which may be a connector, e.g., an audio connector adapted to connect stethoscope device 100 to an external headset port, or to a socket 330 of an external communication device 300. Alternatively, a plug 40 is not required, and instead a wireless connection may be set up between the mobile device 300 and the stethoscope device 100. In this embodiment, stethoscope device 100 may optionally include a WIFI transceiver, Bluetooth transceiver 50 or a Near Field Communication transceiver which can be paired to external mobile device 300.

Plug 40 may include, for example, four electrical contact portions. A first contact portion, when referenced to ground signal, is used for transmitting the physiological sounds from the microphone 30. Two additional contact portions, when referenced to ground, are used for transmitting the call audio, e.g., for right and left stereo audio, to the earpiece or headset 60. The fourth contact may be the ground signal.

In yet another embodiment, stethoscope device 100 may be configured for a local examination by a physician, without exposing the physician to risk for example in a highly contagious biological hazard area. As experienced during the Covid-19 pandemic in 2020, hospital wards that provide care for Covid-19 patients require very strict biological hazard protection for the medical staff. In such environments, it is useful to have a stethoscope device that can optionally be operated by the patient and does not require a physician to expose his ears or face for putting the stethoscope earpieces into them. The following configuration of a stethoscope device may be used —the stethoscope device 100 may include a stethoscope head 10, a microphone 30, a hollow tubing 20 connecting the stethoscope head 10 to the microphone 30, a power supply such as a battery to provide power for the microphone 30, and a speaker though which the physiological sounds may be heard by the examining physician. Alternatively, instead of a speaker, the stethoscope device 100 may include a wireless connection unit which allows wireless connection pairing of a wireless earpiece or headset to stethoscope device 100. In this embodiment, the stethoscope device 100 need not connect to an external communication device, thus does not need to include a plug 40. It is noted that in this embodiment, a power supply unit is required for supplying power to the wireless communication unit. In another embodiment, instead of a speaker, wireless earphones can be paired with device 100 via Bluetooth.

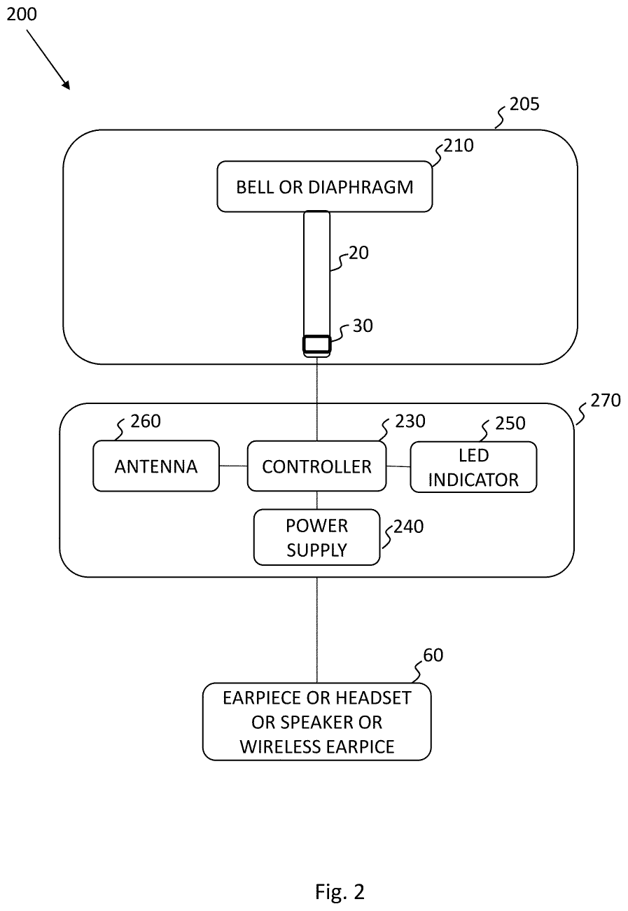

Reference is now made to FIG. 2, which is a block diagram of an example stethoscope device 200 for performing a remote physical examination of a patient, according to another embodiment of the invention. The device 200 may include a stethoscope head 205. The stethoscope head 205 may include or otherwise be coupled to a stethoscope bell 210, formed for placing directly on the skin of a patient and for passing the audio signals, e.g., breathing sounds of lungs inflation and deflation, or heartbeat sounds from the patient's body. The bell 210 may be, for example, a bowl-shaped metal unit which is formed with a sufficient curvature, e.g., sufficiently concave, which allows pick up the physiological sounds in an optimal manner when placing it on the patient's skin. If the curvature of the bell 210 is insufficient, it may pick up noise that originates from the bell touching the skin, in addition to the desired internal physiological sounds. It is noted that in some embodiments, the stethoscope head 205 may be or may include a diaphragm instead of a bell 210. Device 200 further includes a microphone 30 for receiving the physiological sounds passed from bell 210. Stethoscope head 205 may further include a microphone 30 and a tubing 20 coupling the microphone to the bell.

Device 200 comprises a wireless communication unit 270 to wirelessly connect to an external communication device, e.g., device 300 of FIG. 1. The wireless communication unit comprises a controller 230, an antenna 260, and power supply unit (e.g., battery) 240. The wireless communication unit 270 optionally includes a LED indicator 250 for providing LED indications to a user. The microphone 30 is connected via tubing 20 to controller 230, which provides wireless connection capability (e.g., using Bluetooth, NFC or other wireless communication protocols) of the stethoscope device 200 to an external communication device, e.g., a mobile phone, a laptop, a personal computer, a tablet, dedicated device with cellular SIM card, or any other external device. Controller 230 may optionally be operationally connected to an amplifier (not shown), and may allow control of the amplifier, e.g., by a user control button, dial or switch that enables the user to increase or decrease the amplification level. Controller 230 further allows providing indications, e.g., LED (laser emitting diode) indications for providing feedback to the user. Optional LED indications include, for example, an indication that the stethoscope head 205 is paired to an external device 300 (e.g., using Bluetooth or other wireless or near field communication protocols), an indication that the battery power level is low, an indication of an on-going call and examination, etc.

It is noted that in case of using a bell, stethoscope bell 210 may comprise a surrounding ring. The ring may be made of a relatively soft material, e.g., soft rubber or silicon, in order to dampen environmental noise and to stop the stethoscope head 205 from vibrating, making the bell 210 less prone to static electrical noise or external noise interference.

The microphone 30 may be optionally connected to an amplifier (not shown), which is adapted to amplify the electrical signals that are passed via the microphone 30. Microphone 30 may optionally be insulated, using acoustic insulating material such as foam, etc. the insulation layer may be configured surrounding the microphone 30.

Device 200 may include or may be operationally connectable to at least one earpiece or headset 60, which is electronically coupled to the controller 230 and allows the patient to hear the physician's instructions while the remote physical examination is taking place, e.g., when the patient is talking to the physician using a voice or video call. The headset 60 delivers the physician's voice sounds through the voice/video call that is set up between the patient and the physician, which is required for the physician to provide examination instructions during the remote physical examination.

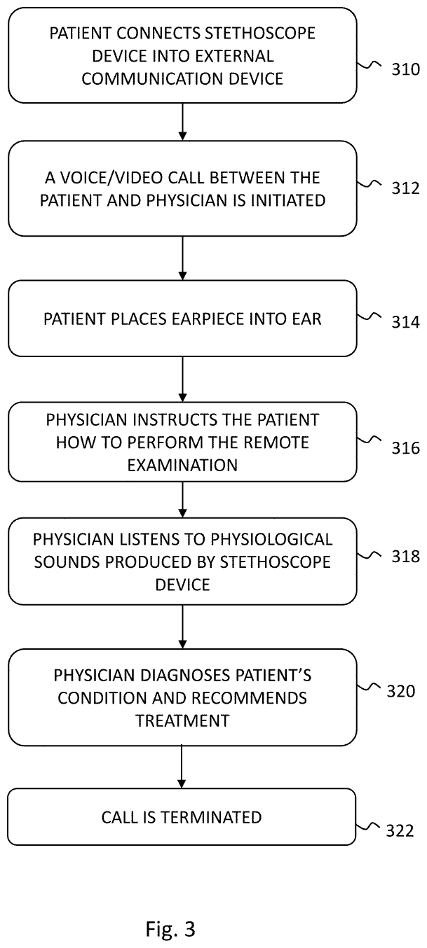

Reference is now made to FIG. 3, which is a flowchart of a method for remote physical examination of a patient as suggested by an embodiment of the invention. It is noted that these steps are not necessarily required to be performed in the order mentioned.

In operation 310, patient connects a stethoscope device into mobile device 300, e.g., stethoscope device 100 or stethoscope device 200 as described in FIGS. 1 and 2 hereinabove. The connection may be a physical connection using an audio input port 330 of the external communication device 300, or using a wireless connection of the external communication device 300. In operation 312, the patient initiates a video call or a voice call with the physician. The video call is preferable as the physician is able to view the patient (or a person assisting the patient) placing the device onto his/her body, and may easily correct as necessary. In operation 314 the patient places earpiece or headset into ears. In operation 316, the physician instructs the patient regarding the examination procedure, e.g., where to place the stethoscope device 100 and how to breath in order to obtain the optimal physiological data during the examination. The physician is able to listen to the physiological sounds (e.g., the breathing sounds/heartbeat sounds) in operation 318. In operation 320 the physician diagnoses the patient's conditions and recommends treatment. The patient follows the physician's instructions and in operation 322, the call is terminated.

It is noted that such method enables the patient's physician to be an integral part of the remote examination process. The remote examination is conducted synchronously with the voice or video call, in real time, and thus the physician does not need an additional phase of, for example, waiting for remote examination data to arrive (e.g., if the data was captured asynchronously then sent for review). The method enables synchronous transmission of the patient's voice, video and physiological sounds data in real time, as well as real time voice and/or video interaction with the physician performing the examination.

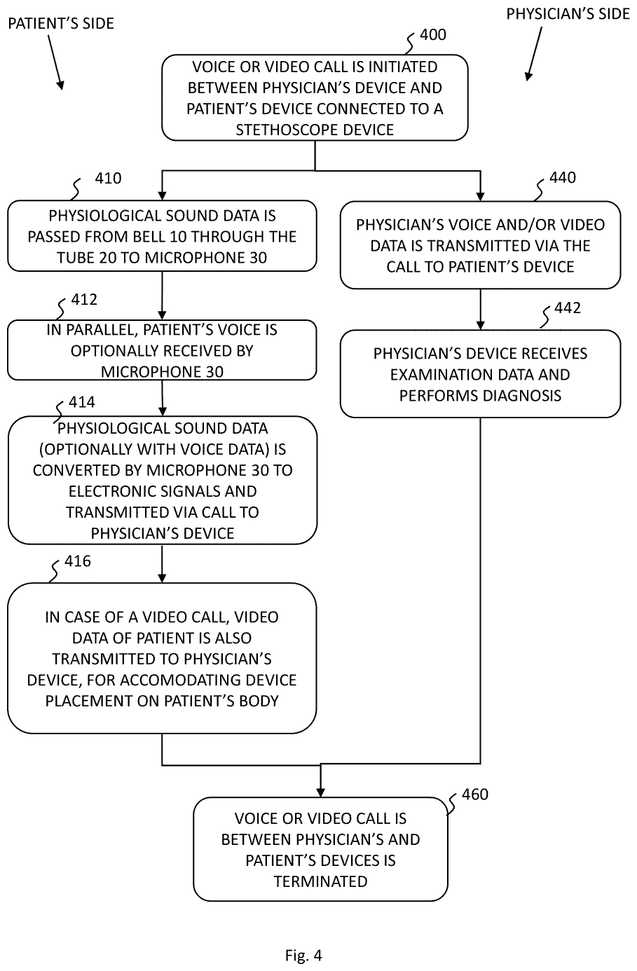

Reference is made to FIG. 4, which is a flowchart of another method for remote physical examination of a patient. It is noted that the described steps are not necessarily required to be performed in the order mentioned. In operation 400, a voice or a video call may be initiated between a patient's communication device (e.g., mobile device 300) and a physician's communication device. The patient's communication device is connected to a stethoscope device, e.g., stethoscope device 100 as shown in FIG. 1. The communication devices appropriate for connecting to the stethoscope device may be selected from mobile phones, tablets, computers connectable to a network, or any other communication device that enables setting up a call via telephony and/or IP networks.

On the patient's side of the call, physiological sound data is received by the stethoscope head 10 and passed through tube 20 to microphone 30 (operation 410). The microphone may also pick up the voice of the patient who may be speaking to the physician (operation 412). In operation 414, the examination sound data obtained by microphone 30 (including the stethoscope sound data and the patient's voice) is converted into electrical signals and transmitted via the call to the physician's device. The physician may respond by speaking with the patient or, in a video call, may show the patient how to position the stethoscope device on his/her body (operation 440). In case of a video call, the examination data including the physiological sound data and the patient's voice and video data, is continuously transmitted to the physician's communication device throughout the call (operation 416). The physician receives the examination data and performs the diagnosis accordingly (operation 442). After concluding the examination, the call is terminated in operation 460.

The physician communicates in a live, online voice call or video session with the patient, guiding the patient throughout the physical examination of the use of the device and placement on the body. The patient listens to the physician through the headset and may communicate back. Patient's vocal communication is transferred to the physician through the headset connected to stethoscope device 100, via microphone 30.

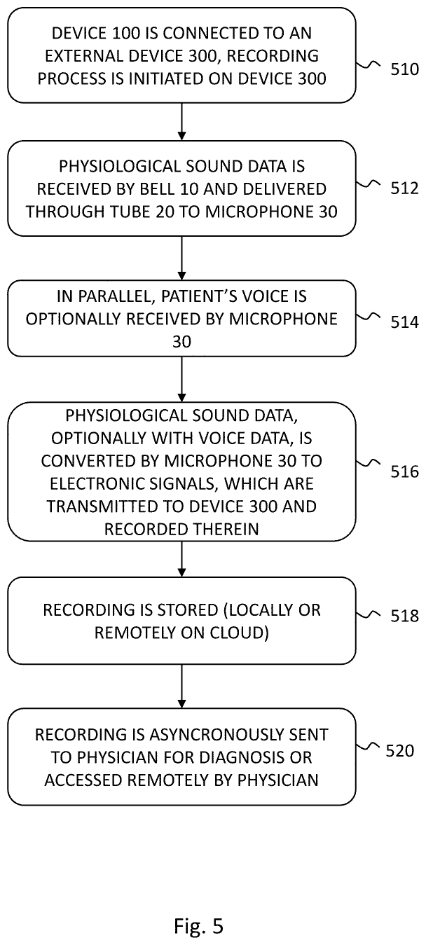

Reference is made to FIG. 5, which is a flowchart of another method for remote physical examination of a patient. It is noted that the described steps are not necessarily required to be performed in the order mentioned. In operation 510, stethoscope device 100 is connected to external device 300. In this embodiment, external communication device 300 comprises recording capability, and the recording process is initiated. In operation 512, physiological sound data is received by stethoscope head 10 and delivered through tube 20 to microphone 30. In parallel, in operation 514, the patient's voice may also be recorded by microphone 30, but the patient need not necessarily speak.

In operation 516 physiological sound data, optionally with the obtained voice data, is converted by microphone 30 to electrical signals, which are transmitted to device 300 and recorded therein. The recording may be stored locally (e.g., using the storage comprised in or coupled to device 300). In some embodiments, the recording may be remotely stored in a cloud network storage unit (operation 518). In operation 520, the recording may be sent to the physician, e.g., by email, file transfer, messaging application, etc. Alternatively, the physician may access the recording in a remote storage location, e.g., by accessing a cloud-based storage unit.

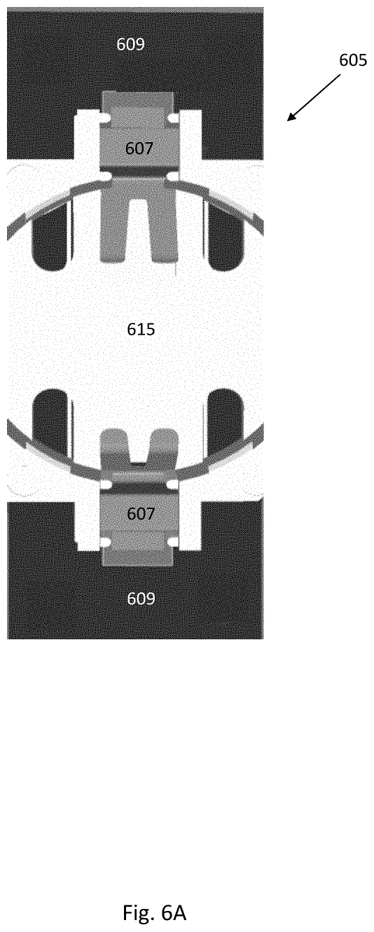

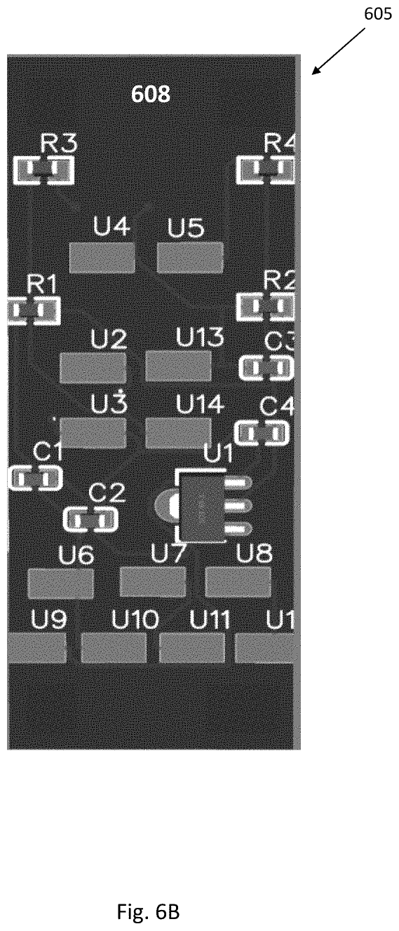

Reference is made to FIGS. 6A and 6B, which illustrate a top view and a bottom view of a PCB which may be used in accordance with an embodiment of the invention. The PCB includes a circuit board or substrate which has a top surface 609 and a bottom surface 608. The PCB 605 may be adapted for electrical impedance matching between stethoscope device 100 to external communication device 300. Top surface 609 may be coupled to a battery housing 615 which is configured to enclose a battery that delivers power supply to the microphone 30. Connector 607 may connect between the battery housing 615 and the Top surface 609. FIG. 6B illustrates a bottom view of the PCB 605 and illustrates various electrical components and contacts. Contacts U2, U3 may be used to connect to an on/off switch for powering the device, in case an external power source such as a battery is used. Contacts U13, U14 may be connected to LED indications to indicate that the battery is activated. Contacts U4, U5 are electrical contacts used for connecting to the microphone 30. Contacts U6, U7, U8 are electrical contacts used for coupling a headset or earpiece to the PCB 605, for connecting the external communication device's 300 audio output to a stereo audio output that is delivered to the headset or earpiece. Contacts U9-U12 are electrical contacts which are used to connect the external communication device's 300 audio input and output to the electrical contact portions of plug 40.

Reference is now made to FIG. 7, which is a diagram of a stethoscope device for remote examination according to an embodiment of the invention. Stethoscope head 10 is attached to tubing 20 which holds microphone 30. Tubing 20 is coupled to housing 715, which is a mechanical enclosure (made of, for example, plastic, metal, or any other appropriate rigid material). Housing 715 encapsulates the PCB of FIG. 6A, including battery housing 615, PCB 605, and the electrical contacts and components assembled on the bottom side of substrate 609, as shown in FIG. 6B. Housing 715 further comprises an on/off switch 721, an audio output port 755 which is connectable to an external headset, speaker, or earpiece/s. Connector 717 is used to maintain the position of microphone 30, e.g., either placed inside tubing 20 or adjacent to the edge of tubing 20. In this embodiment, housing 715 replaces the splitter 54 included in the stethoscope of FIG. 1, and the electrical coupling between the earpiece, external device 300, microphone and battery is performed using the PCB 605.

Many modifications and other embodiments of the invention(s) set forth herein will come to mind to one skilled in the art to which the invention(s) pertain(s) having the benefit of the teachings presented in the foregoing descriptions and the associated drawings. Therefore, it is to be understood that the invention(s) is/are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A stethoscope device for remote physical examination of a patient, comprising:
a stethoscope head formed for placing directly on skin of a patient and adapted to capture physiological and sounds;
a microphone to receive the physiological sounds from the stethoscope head and convert the sounds to electrical signals;
a hollow tube to couple the stethoscope head to the microphone;
a plug or a wireless communication unit connectable to an external communication device of the patient; and
a headset or a headset port to receive audio communication from a remote device.

2. The device of claim 1 wherein the stethoscope head is adapted to obtain vocal sounds in addition to the physiological sounds.

3. The device of claim 1 further comprising a ring surrounding the stethoscope head.

4. The device of claim 1 further comprising a disposable cover which is mountable on the stethoscope head for a single-time use.

5. The device of claim 1 further comprising an insulation layer which acoustically insulates the microphone from external audible interference.

6. The device of claim 1 wherein the plug is connectable to an audio port of the external communication device.

7. The device of claim 1 further comprising a power supply unit coupled to the microphone.

8. The device of claim 7 further comprising a housing adapted to enclose the power supply unit and the microphone.

9. The device of claim 1 wherein the wireless communication unit comprises an antenna and a power supply to provide power to electronic components of the stethoscope device.

10. A stethoscope device for remote physical examination of a patient, comprising:
a stethoscope head formed for placing directly on skin of a patient and adapted to capture physiological sounds;
a microphone to receive the physiological sounds from the stethoscope head and convert the sounds to electrical signals;
a hollow tube configured to couple the stethoscope head to the microphone; and
a power supply unit to provide power to one or more components of the stethoscope device.

11. The device of claim 10 further comprising a wireless communication unit to wirelessly connect the stethoscope device to the patient's external communication device

12. The device of claim 11 further comprising a headset.

13. The device of claim 10 further comprising a headset port.

14. The device of claim 10 further comprising a speaker.

15. The device of claim 10 further comprising an insulation layer which acoustically insulates the microphone from external audible interference.

16. The device of claim 10 wherein the wireless communication unit comprises an antenna, a power supply unit, and a wireless controller, wherein the wireless controller is connected to the microphone, the antenna, and the power supply unit for wirelessly connecting the stethoscope head to an external device.

17. The device of claim 10 further comprising an amplifier to amplify obtained examination data.

18. A method for remote examination of a patient comprising:
providing a stethoscope device comprising a stethoscope head formed for placing directly on skin of a patient, a microphone to obtain physiological sounds and convert the sounds to electrical signals, a hollow tube to couple the stethoscope head to the microphone, a plug connectable to an external communication device and a headset or a headset port;
connecting the stethoscope device to a patient's external communication device;
initiating a voice or video call between the patient's external communication device and a physician's external communication device;
transmitting physiological examination data obtained by the stethoscope device from the patient's external communication device to the physician's external communication device via the call;
receiving at the patient's headset real time instructions from the physician to the patient; and
receiving the physiological examination data at the physician's external communication device.

19. The method of claim 18 wherein the physiological examination data includes physiological sound data and the patient's voice obtained by the stethoscope head.

20. The method of claim 18, wherein the physiological examination data is recorded and stored for use by a physician.
